(19) United States
(12) Patent Application Publication
(10) Pub. No.: US 2025/0164506 A1
(43) Pub. Date: May 22, 2025

(54) SENSING DEVICE FOR PREDICTING THE RISK OF AN COPD AND/OR ASTHMA ATTACK, MEASUREMENT ARRANGEMENT AND METHOD FOR THE MAINTENANCE OF THE SENSING DEVICE

(71) Applicant: BIOINITIALS GMBH, Biel-Benken, BL (CH)

(72) Inventors: Nihat KARAOGLU, Saint-Sulpice (CH); Saso JEZERNIK, Regensdorf (CH); Christian JÄGGI, Biel-Benken (CH); Christoph SCHÄFER, Engelberg (CH); Fabio CIRILLO, Ormalingen (CH); Stefan STÜBINGER, Binningen (CH); Stefan SCHAFFNER, Münchenstein (CH)

(21) Appl. No.: 18/833,994

(22) PCT Filed: Jan. 27, 2023
(86) PCT No.: PCT/EP2023/052017

§ 371 Date: Jul. 29, 2024

Related U.S. Application Data
(60) Provisional application No. 63/304,650, filed on Jan. 30, 2022.

(51) Int. Cl. G01N 33/68; A61B 10/00; G16H 50/20

(52) U.S. Cl. CPC G01N 33/6893; A61B 10/0051; G16H 50/20; G01N 2800/122; G01N 2800/50

(57) ABSTRACT

A sensing device is used to determine the risk and/or probability of an COPD-attack and/or asthma attack of a patient. The sensing device includes a collection device for the non-invasive collection of a biological sample and a biosensor for determining the existence and/or the amount of a biomarker in the biological sample, preferably without the need for sample preparation. The device also includes a control and evaluation device that determines a risk and/or probability of an COPD and/or asthma-attack based on the existence and/or measured amount; and a measuring arrangement and a method for the maintenance of said sensing device or measurement arrangement, and an output unit that outputs the risk/probability.

### BACKGROUND AND SUMMARY OF THE INVENTION

Exemplary embodiments of the invention relate to a sensing device and a method for the predicting the risk of a Chronic obstructive pulmonary disease (COPD) and/or an asthma attack, as well as to a measurement arrangement and a method for the maintenance of the sensing device.

Chronic obstructive pulmonary disease (COPD) is a variation of a progressive lung disease defined by long-term respiratory symptoms and limitation of the airflow. The symptoms characterizing the disease includes cough and shortness of breath, which could be produced by the mucus.

Asthma is a variation of a COPD and a long-term, chronic inflammatory disease of the airways of the lungs. In asthma the airways narrow, swell and may produce extra mucus. This makes breathing difficult and triggers coughing, wheezing, and shortness of breath. Asthma is characterized by variable and recurring symptoms, reversible airflow obstruction, and easily triggered bronchospasms. It is thought to be caused by a combination of genetic and environmental factors. Environmental factors include exposure to air pollution and allergens. Other potential triggers include medications such as aspirin and beta blockers.

Symptoms include episodes of wheezing, coughing, chest tightness, and shortness of breath. These may occur a few times a day or a few times per week. Asthma condition can also culminate in an asthmatic attack, a severe and life-threatening event. During an asthma attack, also called an asthma exacerbation, the airways become swollen and constricted. The muscles around the airways contract and the airways produce extra mucus, both effects causing the breathing (bronchial) tubes to narrow and to severely restrict breathing in a life-threatening way.

Currently, there is no cure for asthma patients. In 2019 asthma affected approximately 262 million people and caused approximately 461,000 deaths. Most of the deaths occurred in the developing world. Asthma often begins in childhood, and the worldwide incidence and prevalence rates have increased significantly since the 1960s. It has been reported that 1 in 10 children has asthma and asthma is among the top 10 of the most frequent causes of death worldwide.

Diagnosis is usually based on the recognition of sets/pattern of symptoms, spirometry testing, and induced bronchoconstriction lung function testing. Diagnosis is typically also confirmed by expected responses to pharmacological treatments.

Asthma is treatable as a chronic condition and alleviated with chronically taken medications. Severity of the symptoms can be reduced by avoiding triggers, such as allergens and respiratory irritants, and suppressed with the use of inhaled corticosteroids. Long-acting beta agonists (LABA) or antileukotriene agents may be used in addition to inhaled corticosteroids if asthma symptoms remain uncontrolled. Treatment of rapidly worsening symptoms is usually with an inhaled short-acting beta-2 agonist such as salbutamol and corticosteroids taken by mouth. In very severe cases and during or following an asthma attack, intravenous corticosteroids, magnesium sulfate, and hospitalization may be required.

Management of asthma aims to control the occurrence and frequency of asthmatic attacks and tries to ensure a good quality of living. But at this moment there is no established mechanism to predict and prevent an asthma attack. This makes it very difficult for the patients to plan their lives around the disease, and methods are needed to estimate the risk of a sudden attack that might be life-threatening. Fatal asthma attacks are also frequently observed with patients who are unaware of the possibility of experiencing a fatal attack. Fatality is more likely when the person undergoing an attack is alone (unattended).

In 2019 asthma affected approximately 262 million people and caused approximately 461,000 deaths worldwide. Most of the deaths occurred in the developing world. Asthma often begins in childhood, and the rates have increased significantly since the 1960s. Especially in babies and children where initial symptoms may not be recognized, a sudden attack may cause loss of life. Parents often notice when the attack is developing hearing a wheezing, coughing and shortness of the breath. However, when the child, or a young adult or an elderly person is unattended, such as during night sleep, a sudden onset of an attack may put the life of the person in danger.

EP3367101A1 discloses a method in which biomarker IL-17 (alone or together with other specific biomarkers) present in blood, serum, or plasma is measured to predict an asthmatic attack. However, there are major drawbacks applying this approach, which make the method not well suited for daily management of asthma.

In this prior art, the biomarker is derived from blood either serum or plasma, therefore the method disclosed in the prior art is invasive. Thus, the patient experiences discomfort each time a blood sample is taken.

In this prior art, the main targeted biomarker is IL-17 and some supplemental biomarkers are of interest and disclosed by the following wording “measuring IL-17 and at least one biomarker selected from the group consisting of IL-10, IL-4, IL-25, IL-9 and IFN-y.”. However, biomarkers that were linked to asthma and the risk of asthma attacks are not targeted. The measurement of IL-17 is only possible by a person skilled in the art, e.g., lab technician, however the patient or end-user remains unable to do so. The detection further requires special lab equipment such as a microplate fluorescence reader and is therefore not practical to use at the point of care by an unskilled person.

The prediction accuracy is 12 months. Hence there is no practical use of the prior art for predicting attacks that might happen fast, in a small prediction time-horizon.

Exemplary embodiments of the invention are directed to providing a sensing device and a method for the prediction of the risk and possibility of a COPD and/or asthma attack.

Disclosed is a device and a method for predicting the risk and possibility of a COPD and/or asthma attack and informing the patient, caregiver or healthcare professionals about the risk and prediction.

This is achieved by continuously and/or frequently monitoring and processing the levels and changes in biomarkers related to the asthma disease.

The invention comprises a device for collecting oral sample from the patient, a method for measuring the level of biomarkers in the sample, and an algorithm for predicting the risk and possibility of an attack and a system for informing the users so that they can take precautionary or counter-measures to avoid such an attack.

It has been observed that chances of experiencing asthma symptoms and attacks are higher during sleep, called nocturnal or night-time asthma events/condition. In case of a sudden-onset, a nocturnal attack may be fatal, ranging from babies, children, to adults and elderly. Different users belonging to different age segments might utilize different preferred devices with integrated sensing for monitoring of risk of asthma attacks and prevention of exacerbations. Babies would preferably use pacifier or feeding-bottle based devices, children and adult's toothbrush devices, and elderly other types of oral implements or even solutions with prosthetic devices with integrated sensing.

It has been shown that certain biomarkers are related to the asthmatic disease.

The current invention solves the problem of predicting exacerbations in less than 48 hours, such that the estimated risks are calculated based on a novel noninvasive sample collection, a novel biomarker sensing method, and a novel device; results of the analysis of current and historical data and data patterns are presented to the user and medical health professionals on a daily basis with one or more daily updates of the estimated risks of asthmatic attacks and current asthma condition.

Based on the updates, patients and caregivers are enabled to take countermeasures in order to counteract high risks and effectively prevent asthma attacks in a controlled, conscious way.

The benefits range from better chronic disease management of asthma to preventing severe asthma attacks and complications, and, ultimately, possible prevention of loss of life.

It is advantageous—if the device is adapted to repeat a measurement multiple-times per day by a non-invasive sensing of biomarkers linked to asthma condition and asthma exacerbations and to the risks and probability of occurrence of asthma exacerbations.

The current invention has its focus on a novel set of biomarkers comprising immunoglobulins IgE, IgA, interleukins, and other relevant biomarkers like CRP, TSLP and Lysozyme.

A further focus is on the integration of a Biomarkers-sensor herein also referred to as a biosensor for the determination of the existence and/or the amount of a biomarker in the biological sample. The amount could for example be the content, also referred to as concentration, of a specific biomarker or multiple biomarkers in the sample.

In a preferred embodiment there is no need for a sample preparation in order to detect the biomarkers by the biosensor. In other words—the biosensor is provided for a direct determination of the biomarker or the amount of this biomarker within the sample, without any further sample preparation. Of course, there are other embodiment according to the invention where the sample preparation, such as the addition of a marker, such as a fluorescence marker, is provided before the measurement by the biosensor.

In an oral implement device or device that is used in the oral cavity by different users including elderly and babies. The present invention addresses the problems listed above in order to offer a practical method that is useful in daily management of asthma.

In a first preferred aspect of the invention the biomarkers are collected from oral excretions or secretions (saliva), in a non-invasive way. Thus, the inventive device is provided with a sampling section, also referred to as collection device, preferably provided with a sampling channel for the drawing of a sample of an excretions or secretion. The collection device is designed for the non-invasive collection of a biological sample, such as saliva.

The present invention addresses the problems listed above in order to offer a practical inventive method that is useful in daily management of asthma.

The sample is processed by the inventive device, which is preferably a portable device, such as a handheld. The handheld can be operated by the patient or caregiver by activating the portable device.

The sensor might be adapted for measuring selectively the concentration of biomarkers from a group consisting of: IgE, IgA, CRP, TSLP, lysozyme, interleukins IL4, IL5, IL6, IL-10, IL-13, IL-17, IL-19, IL-25 and/or IL32.

The current invention comprises a non-invasive method and device for collecting biological samples from a patient.

The sample can be oral excretion such as sputum or oral secretion, such as saliva.

There are different possibilities for collecting the oral excretion and/or secretion. For this purpose, the inventive sensing device is provided with a collection device. It can be sampled through an extra-oral vessel such as tube or vial, where the patient delivers the sample into channels or chambers of the collection device. The collection device can be part of and especially integrated in the inventive device.

A further possibility of sampling is through an intra-oral implement such as a sampling stick, a pacifier device, a toothbrush device, a feeding-bottle device, a dental equipment, preferably for the application at and/or replacement of teeth such as a bracket, bridge and/or a dental bracelet and/or a dental implant.

The device, especially the sensing device, according to the invention is provided with a control- and evaluation device adapted to measure the levels of biomarkers in the sample in a quantitative way. The control- and evaluation device can be provided with a processing unit and with a data storage. The data storage can be provided with the aforementioned algorithm and with a computer program for carrying out the aforementioned inventive method.

The sensing mentioned intra-oral implements or other external devices.

The sensing device, especially the biosensor, can be provided to determine or use at least one or more biochemical properties of the sample.

The sensing device, especially the biosensor, can be provided to determine or use at least one or more immuno-chromatographic properties of the sample.

The sensing device, especially the biosensor, can be provided to determine or use at least one or more electrochemical properties of the sample after a reaction with reagents or assays.

The sensing device, especially the biosensor, can be provided to determine or use at least one or more optoelectronic properties of the sample, especially additionally to the measurement of the concentration of biomarkers.

The sensing device can be further provided with a turbidity sensor.

The control- and evaluation device can be provided to use electrical or magnetic properties of the sample, especially additionally to the measurement

The sensing device may be provided to use any combination of the aforementioned measurements. Especially the sensing device can be provided with docking ports to exchange sensors that are not suitable for the person, which are defective or have reached their end-of-life. Babies may need other biosensors that detect other biomarkers or other sensors in general than elderly people. The sensing device can be used as a platform for the prediction of different types of COPD depending on biological factors of the patient, such as the age.

The biomarkers for the prediction of the risk of a COPD-Attack that are measured and tested by the inventive sensing device are at least one or a combination of the compounds of the following group: IgE, IgA, CRP, lysozyme and/or Interleukins such as IL4, IL5, IL6, IL10, IL-13, IL17, IL-19, IL25, IL32, TSLP.

The algorithm used by the control and evaluation device predicts the clinical presentation of the disease based on the measurements, as early as possible.

The prediction of a COPD attack can detect a risk before any audible and/or visible symptoms of the patient occur.

The prediction can be done based on advanced signal and data processing of the biomarker data and preferably further data determined by other sensor devices, such as conductivity or the like. The data processing can be designed based on historic data processing and analysis or advanced methods such as model building, machine learning, and/or advanced methods of detection, prediction, classification and/or pattern recognition.

A further subject of the invention is a measuring arrangement comprising one or several inventive sensing devices. The sensing devices are electronic devices.

They can be provided with bidirectional wireless capabilities, mobile computing devices, and/or networking systems or cloud-based systems and/or software applications, which inform the patient, caregivers or healthcare professionals of the risk and/or with a probability of an upcoming asthmatic attack. This information can preferably be provided in a continuous manner.

The measuring arrangement is used to calculate an aggregate Risk Score for an upcoming COPD and/or asthma, exacerbation, with which the patient takes a preventive action, such as preparing or taking medication or informing a HCP or a caregiver.

The sensing device might be adapted to calculate and communicates a Risk Score for an upcoming COPD and/or asthma exacerbation using a specified software algorithm and/or based on machine learning.

The inventive sensing device or the measuring arrangement may be provided with an analyzing program that collects data over longer periods of time, such as one or more months. This way it may improve the chronic management of a COPD and/or asthmatic condition.

The aforementioned measuring arrangement is provided to perform a non-invasive method for collecting biological samples from a patient from the oral cavity by using a collection device for oral secretions.

The collection device may be an integrated or exchangeable part of the inventive sensing device and can be an extra-oral vessel such as a tube or a vial, where the patient delivers the sample into the vessel. The same applies for the biosensor or a sensing unit comprising the biosensor.

Additionally, or alternatively, the collection device can be or be provided with a sampling stick where the sample is collected from the oral cavity or a pacifier or a toothbrush, or a dental device such as a bracket, a bridge, a bracelet and/or a dental implant.

The sensing device measures the levels of biomarkers in the sample in a quantitative way. The measuring arrangement with the sensing device or preferably the sensing device may be provided with a control and/or evaluation device which is provided to
evaluate the biochemical properties of the sample;
evaluate the immunochromatographic properties of the sample;
evaluate the electrochemical properties of the sample after a reaction with assays;
evaluate optical or electrooptical properties of the sample;
evaluate electrical or magnetic properties of the sample and/or
evaluate any of the above combinations of properties of the sample.

The inventive sensing method may also comprise one or more of the evaluations. The evaluations might be combined in a preferable embodiment of the invention.

The sensing device is provided such that the device uses the sensing method together with the aforementioned one or more data processing algorithm. The sensing device is further provided to inform the user about the asthma condition and the risk and probability of an asthma attack.

The device is further provided to predict the clinical presentation, the state of the disease, the risk and/or the probability of a COPD attack and/or exacerbation and/or an asthma attack and/or exacerbation. The provision of the device is based on the measurements and processing of oral sample properties and biomarker levels contained there-in.

The data processing algorithm is based on advanced signal and data detection, classification, prediction, pattern recognition, machine learning, which can learn, differentiate and/or predict the change patterns in biomarkers and its relation to COPD attacks and/or asthma attacks, for a specific individual or groups of patients.

Alternatively, or additionally, the algorithm can be based on statistical or stochastic or neural network or machine learning modeling or other type of model-based algorithmic design.

The measuring arrangement with the sensing device informs the patient, caregivers or healthcare professionals with or without any audible, visible symptoms, with enough accuracy and foresight about the risk and probability of a building-up or imminent COPD attack and/or an asthma attack.

The measuring arrangement is further provided to provide informs whether the sensing method is viable or must be readjusted.

A further object of the invention is a method for the maintenance of the sensing device and/or the measuring arrangement. This method comprises the following steps:
A Sampling a biological sample by a non-invasive method from an oral cavity of a patient;
B Determining the content of a biomarker in the sample by a measurement in a sensing method;
C Comparing the results of step B with one or more previous measurements or with a predefined threshold;
D Initiating a cleaning of and/or providing an information about an exchange of the collection device and/or the biosensor and/or a sensing unit comprising the biosensor.

The sensing method is the sensing method which has been previously described and which is also inventive, as far as it is allowable within the jurisdiction the invention seeks protection.

The control- and evaluation devices are provided to perform the sensing method. This provision can be realized for example by one or more computer programs and/or electronic circuits. The computer programs can be stored in a storage device such a RAM or ROM of the control- and evaluation device and can be performed by one or more CPU's.

### BRIEF DESCRIPTION OF THE DRAWING FIGURES

The inventive measuring arrangement, the sensing device and/or the inventive method will be further explained in the context of different embodiments and with reference to Figures. They show:

FIG. 1 a sectional view of a toothbrush-like device as an embodiment for an inventive sensing device.

FIG. 2 a sectional view of a feeding bottle as an embodiment for an inventive sensing device; and

FIG. 3 a sectional view of a pacifier as an embodiment for an inventive sensing device.

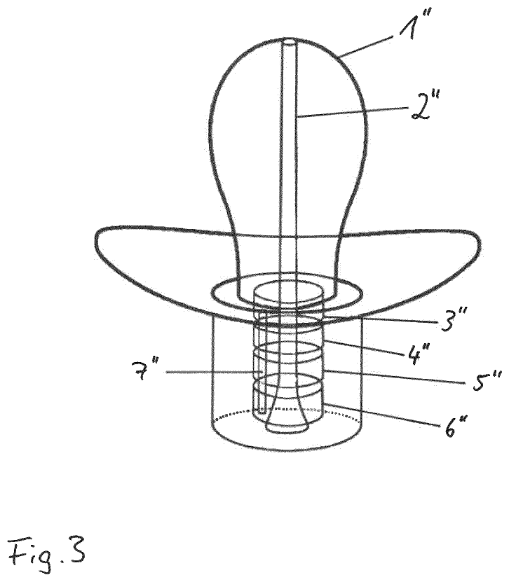

### DETAILED DESCRIPTION

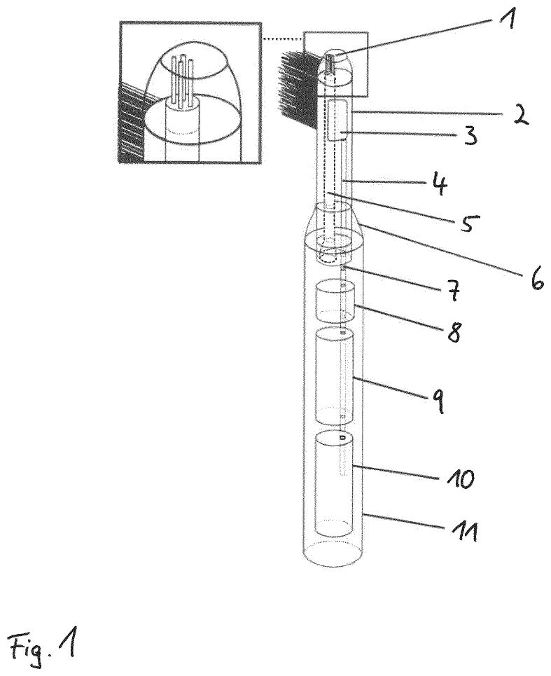

FIG. 1 illustrates a particular embodiment in which the sensing device 11 is a toothbrush as an intra-oral implement with an exchangeable tip salivary as a collection and sensing part preferably comprising a sensor, especially a biosensor for the determination of a content of a specific biomarker in the biological sample, especially the collected saliva sample. The determination of the content may be the determination of a value and the comparison of the value with a threshold. It may also be a concentration measurement, wherein the concentration is compared with multiple thresholds, each comprising a different risk and/or probability for a COPD and/or asthma attack. The determination comprises at least the determination of the content of only one biomarker sample. For reasons of higher accuracy and/or redundancy the determination may comprise the determination of the content of 2 or more, thus multiple, biomarkers in the sample. Thus, there could be more than one biosensor for the determination of a content of a specific biomarker in the biological sample integrated in the inventive device or the only one biosensor may determine the content of more than one specific biomarker in the biological sample.

When the sensing device is a toothbrush, a sampling or collection device with an saliva inlet 1 can be integrated in an exchangeable toothbrush head 2, which is used as oral excretion or secretion collection component. The sampling or collection device can comprise a set of one or more fluidic channels and/or capillary tubes used in collecting saliva from the toothbrush bristle area or the top of the tip and guiding the saliva to a collection chamber where it gets in contact with the biosensor for the determination of a content of a specific biomarker in the biological sample 3.

In one particular embodiment, not shown in FIG. 1, a modification of the toothbrush of FIG. 1 with capillary tubes can be used. At each use and salivary collection, one of the capillary tubes can be opened by the user, while the other capillary tubes remain blocked by a special multi-plug system. The capillary tubes can be unplugged one-by-one for their consecutive use. Alternatively, or additionally, the toothbrush head can be removed after usage and the saliva tube can be rinsed with water or another suitable fluid.

The biosensor for the determination of a content of a specific biomarker in the biological sample 3 is connected by electric communication means 4, such as a cable, to the sensor electronics via the connection unit and an electric connector 7, used for power transfer and communication. The exchangeable head component is connected to the base component via a suitable interface by a connector unit 6.

The toothbrush handle may contain the control- and evaluation device 8, which can preferably be provided as an embedded electronic system with one or more CPUs that are processing several input sensing channels that were conditioned with analog electronics and digitalized. The control- and evaluation device 8 further comprises a data storage where the aforementioned algorithm and preferably a computer program product for the execution of the inventive method is stored.

The control- and evaluation device 8 can be positioned in the toothbrush handle but it is alternatively possible that the control- and evaluation device 8, or at least parts of it, are positioned in an external device outside the toothbrush, such as an electronically mobile device, like a mobile phone or a laptop. In this context the mobile device is defined to be part of an inventive measuring arrangement comprising the sensing device 100 and the external device, for example a mobile device.

In the same context the inventive sensing device and/or an external device of the measuring arrangement can be provided with an output device, also referred to as a output unit, such as a screen, a scaling of the risk, such as 0, 10%, 20% . . . 100%, or a light signal and/or an acoustic device or even a vibration device which can provide the user with information and/or warning about the risk of a COPD- and/or asthma attack. The output device preferably gives an information of a risk, warning and/or probability of a COPD- and/or asthma attack within less than the next 48 hours, more preferably less than 24 hours, after the measurement.

In this context the output device can be provided to produce a wake-up signal, such as an acoustic signal, a vibration signal or even a heat signal to the user if a high risk of a COPD an/or asthma is determined in the sleeping phase of the user.

The output device can be integrated inside the sensing device or be part of an external device being part of the inventive measuring arrangement.

The processed signals and data samples are then transferred to a communication unit 9—which preferably comprises bidirectional wireless communication means that can be realized, for example, by different wireless communication technologies such as LoRA, Bluetooth, 4G or 5G, preferably with corresponding low-power radios & chipsets.

The electronics is powered by a power electronics unit 10 provided with an accumulator and/or batteries, preferably rechargeable batteries. The power electronics unit allows also for wireless power transfer to the toothbrush in order to recharge the integrated batteries. The biosensor for the determination of an existence and/or amount of a specific biomarker in the biological sample can also contain means for removing the excess saliva and for allowing for a saliva wash-out through additional channels that pass across the sensing unit. One or more additional saliva channels 5, comprising an inlet and outlet, are needed for the purpose of sampling saliva and removing excess saliva.

Priming of these saliva channels 5, herein also referred to as tubes, can be accomplished with priming with water or air or other fluid. This can be done for example by a small syringe that the user fills-up with air and then presses the air out after connecting the syringe or a suitable syringe connector with the saliva inlet 1 or saliva channel 5.

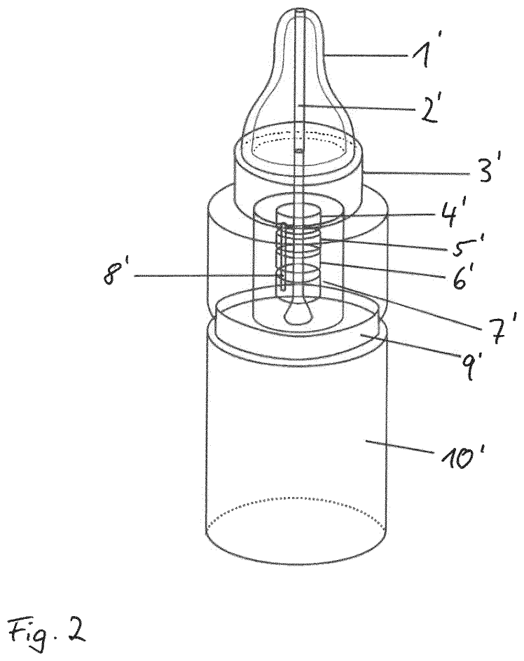

FIG. 2 discloses a feeding bottle as a second embodiment of an inventive sensing device 1′ according to the invention. The inventive sensing device is a feeding-bottle device with an exchangeable tip salivary collection and sensing part comprising the biosensor for the determination of the existence and/or the amount of a specific biomarker in the biological sample 3′.

When the sensing device 1′ is a feeding-bottle, this functional unit comprises an exchangeable nipple component, with an integrated saliva tube 2′, which is used as oral excretion or secretion collection component. The nipple part used for oral suction and collection of saliva is attached to a screw cap 3′, which connects to the feeding bottle with integrating sensing unit 4′, may contain various biosensors for the determination of an existence and/or amount of a specific biomarker in the biological samples for different biomarkers or other sensors such as sensors for measuring the conductivity. The sensing unit 4′ is further provided with a control- and evaluation device 5′ for the signal and data processing in one or several CPUs. The sensing unit 4′ may be further provided with a communication unit 6′, preferably with a wireless communication electronics with chipsets and/or radios. The sensing unit 4′ may be further provided with a power supply 7′, preferably containing one or more of the power electronics components such as antennas/coils, PU chipsets and/or batteries.

Electronics and device components can preferably be connected each to other by means of a communication cord 8′ and/or a connector device 9′. The housing 10′ of the sensing device 1 can preferably be a real bottle for the feeding of babies or a dummy-bottle without any feeding function but with the form of a feeding-bottle.

In the third embodiment of the invention the sensing device 1″ is a pacifier. The sensing device 1″ could therefore be provided with an exchangeable tip salivary collection and a sensing unit 4″, having the same parts as the sensing device 1′ described in FIG. 2.

Although the invention has been illustrated and described in detail by way of preferred embodiments, the invention is not limited by the examples disclosed, and other variations can be derived from these by the person skilled in the art without leaving the scope of the invention. It is therefore clear that there is a plurality of possible variations. It is also clear that embodiments stated by way of example are only really examples that are not to be seen as limiting the scope, application possibilities or configuration of the invention in any way. In fact, the preceding description and the description of the figures enable the person skilled in the art to implement the exemplary embodiments in concrete manner, wherein, with the knowledge of the disclosed inventive concept, the person skilled in the art is able to undertake various changes, for example, with regard to the functioning or arrangement of individual elements stated in an exemplary embodiment without leaving the scope of the invention, which is defined by the claims and their legal equivalents, such as further explanations in the description.

1-65. (canceled)

66. A measuring arrangement for determining risk or probability of a COPD- or asthma-attack of a patient, the measuring arrangement comprising:
a collection device configured to non-invasively collect a biological sample;
a biosensor configured to determine existence or an amount of a biomarker in the biological sample without requiring preparation of the sample;
a control and evaluation device configured to determine the risk or probability of the COPD- or asthma-attack based on existence or a measured amount of at least one of the biomarker; and
an output unit configured to output
the determined risk or probability of the COPD- or asthma-attack, or
a warning about the determined risk or probability of the COPD or asthma-attack.

67. The measuring arrangement of claim 66, further comprising:
a sensing device comprising at least the collection device and the biosensor, wherein the sensing device is a hand-held or a stand-alone device.

68. The measuring arrangement of claim 67, further comprising:
a device, external to the sensing device, comprising at least the control and evaluation device or the output unit.

69. The measuring arrangement of claim 66, wherein the biosensor is provided for determining biomarkers selected from a group consisting of IgE, IgA, CRP, TSLP, lysozyme, or that the biosensor is provided for the determination of an interleukin.

70. The measuring arrangement of claim 66, wherein the collection device is configured to collect oral excretions or secretions.

71. The measuring arrangement of claim 66, wherein
the collection device comprises a sampling stick or the sensing device comprises a housing;
the collection device or the housing is a pacifier or feeding bottle for babies, the sensing device is a toothbrush, or the sensing device is a dental device, which is one or more brackets, a bridge, a bracelet, an exchangeable or permanent dental brace, or a dental implant.

72. The measuring arrangement of claim 66, wherein the collection device comprises a vessel, wherein the collection device further comprises a channel or tube configured to deliver oral excretions or secretions from a surface of the sensing device to the vessel, and the vessel is exchangeably arranged inside the sensing device.

73. The measuring arrangement of claim 68, wherein the sensing device comprises a communication unit configured to communicate with the device external to the sensing device.

74. The measuring arrangement of claim 66, wherein the control and evaluation device is configured to use biochemical properties, immuno-chromatographic properties, electrochemical properties, optoelectronic properties, electrical properties, or magnetic properties of the biological sample to determine the risk or probability of the COPD- or asthma-attack.

75. The measuring arrangement of claim 66, wherein
the control and evaluation device is configured to use a data processing algorithm, which is designed based on historic data processing and analysis, model building, machine learning, or methods of detection, prediction, classification, or pattern recognition,
the measuring arrangement configured to use and communicate with an aggregate risk score for an upcoming COPD or asthma exacerbation,
the control and evaluation device is configured to use an analyzing program that collects data over a period of time measured in months,
the control and evaluation device is configured to perform a noninvasive method for collecting biological samples from a patient from an oral cavity using a collection device for oral excretions or secretions, or
the control and evaluation device configured to detect or inform whether the detection is viable or the detection must be readjusted.

76. The measuring arrangement of claim 66, wherein the control and evaluation device is configured to:
evaluate the biochemical properties of the biological sample;
evaluate the immunochromatographic properties of the biological sample;
evaluate electrochemical properties of the biological sample after a reaction with assays;
evaluate optical or electrooptical properties of the biological sample;
evaluate electrical or magnetic properties of the biological sample;
predict clinical presentation, state of a disease, the risk or the probability of the COPD or the asthma attack.

77. The measuring arrangement of claim 75, wherein
the data processing algorithm is based on signal and data detection, classification, prediction, pattern recognition, or machine learning, which can learn, differentiate, or predict change patterns in biomarkers and a relation of the change patterns to COPD or asthma attacks for a specific individual or groups of patients, or
the data processing algorithm is based on statistical, stochastic, neural network, or machine learning modeling.

78. The measuring arrangement of claim 66, wherein the measuring arrangement is configured to inform the patient, care-givers, or healthcare professionals with and without the patient having any audible, visible symptoms, about a risk and probability of a building-up or imminent COPD- or asthma-attack.

79. The measuring arrangement of claim 68, wherein the collection device, the biosensor, or a sensing unit comprising the biosensor are exchangeable parts of the sensing device.

80. A method maintaining a measuring arrangement for determining risk or probability of a COPD- or asthma-attack of a patient, the measuring arrangement comprising a collection device; a biosensor; a control and evaluation device configured to determine the risk or probability of the COPD- or asthma-attack based on existence or a measured amount of at least one of the biomarker; and an output unit configured to output the determined risk or probability of the COPD- or asthma-attack, or a warning about the determined risk or probability of the COPD or asthma-attack, the method comprising:
A. sampling, by the collection device, a biological sample by a non-invasive method from an oral cavity of the patient;
B. determining, by the biosensor, existence or an amount of a biomarker in the biological sample, without preparing the biological sample;
C. comparing results of step B with one or more previous measurements or with a predefined threshold; and
D. initiating cleaning of or providing an information about an exchange of the collection device, the biosensor, or a sensing unit comprising the biosensor.
